(19) United States
(12) Patent Application Publication
(10) Pub. No.: US 2008/0214658 A1
(43) Pub. Date: Sep. 4, 2008

(54) Composition Comprising Isoorientin for Suppressing Histamine

(75) Inventors: Sung-Sick Woo, Seoul (KR); Dong-Seon Kim, Daejeon (KR); Seon-Gil Do, Chungcheongbuk-do (KR); Young-Chul Lee, Daejeon (KR); Mi-Sun Oh, Chungcheongnam-do (KR); Ji-Min Cha, Seoul (KR); Tae-Hyung Jo, Gyeonggi-do (KR)

Correspondence Address: BIRCH STEWART KOLASCH & BIRCH, PO BOX 747, FALLS CHURCH, VA, 22040-0747, US

(73) Assignee: UNIGEN, INC., Cheonan-si (KR)

(21) Appl. No.: 11/908,927

(22) PCT Filed: Mar. 17, 2006
(86) PCT No.: PCT/KR2006/000984

§ 371 Date: Feb. 22, 2008

(30) Foreign Application Priority Data
Mar. 18, 2005 (KR) 10-2005-0022772

(51) Int. Cl. A61K 31/352; A61P 43/00

(52) U.S. Cl. 514/456

(57) ABSTRACT

The present invention relates to a pharmaceutical composition for the prevention or treatment of diseases mediated by excessive histamine comprising naturally-derived isoorientin, a use of isoorientin for the manufacture of a medicament for the prevention or treatment of diseases mediated by excessive histamine, and a method for preventing or treating diseases mediated by excessive histamine comprising administering a therapeutically effective amount of isoorientin to a subject. The composition, use and method of the present invention show excellent histamine suppression effects, and so can be used for the prevention or treatment of various kinds of allergic disease, atopic disease, inflammatory disease, skin disease, hyperacidity and nervous system disorder.

### TECHNICAL FIELD

The present invention relates to a pharmaceutical composition for the prevention or treatment of diseases mediated by excessive histamine comprising isoorientin as an active ingredient, a use of isoorientin for the manufacture of a medicament for the prevention or treatment of diseases mediated by excessive histamine, and a method for preventing or treating diseases mediated by excessive histamine in a subject, comprising administering a therapeutically effective amount of isoorientin to the subject.

### BACKGROUND ART

Histamine is a physiologically active substance which is present in blood and various kinds of tissue. Structurally, histamine is also referred as aminoethyl imidazole wherein imidazole ring and amine group are attached to two methylene groups. Histamine can be found in almost all tissues of animal, and is even present in various kinds of toxin, bacteria or plant. Skin, bronchus, intestinal mucosa, etc. contain an abundance of histamine. In blood, basophil contains an abundance of histamine. These cells containing histamine can synthesize histamine by L-histidine decarboxylase from histidine. Non-mast cell in epidermis, gastric mucosa, nerve cell in the central nervous system, etc. also can synthesize histamine.

In the human body, histamine is metabolized in two pathways.

In the main pathway, imidazole ring is converted into N-methylhistamine by N-methyltransferase, and then the N-methylhistamine is converted into N-methylimidazole acetic acid by amonoamine oxidase.

In the other pathway, histamine is oxidatively deaminated by non-specific diamine oxidase. Metabolite of histamine is almost inert, and excreted by urine.

Histamine is known to induce allergy, secrete gastric acid, and function as neurotransmitter in the central nervous system [Corrado M E et al., Arzneimittelforschung, 54(10) 660-5, 2004, Salmun L M., Expert Opin Investig Drugs, 11(2) 259-73, 2002, Scannell R T et al., Mini Rev Med. Chem., 4(9) 923-33, 2004, Kapp A et al., J Drugs Dermatol., 3(6) 632-9, 200. Orzechowski R F et al., Eur J. Pharmacol., 506(3) 257-64, 2005].

First, reviewing the role of histamine in allergy reaction, upon exposure to antigen, antibody (IgE) is produced, which then attaches to a surface of mast cell and basophil to cause histamine release via membrane-phosphorylation. Histamine is a finished form stored in mast cell. Thus, when antigen interacts with IgE antibody in the surface of mast cell, it is released. Phospholipase A₂ is also activated, and so platelet activation factor (PAF), or metabolite of arachidonate such as prostaglandin, leukotriene D₄, etc. is produced and released along with histamine.

Second, pneumogastric nerves or gastrin may accelerate gastric acid secretion, but histamine is the most important substance which regulates gastric acid secretion. When H₂ receptor blocking drug is used, acid secretion by acetylcholine or gastrin as well as acid secretion by histamine are all blocked. Thus, histamine is considered functioning as a final mediator in physiological acid secretion mechanism.

Lastly, reviewing the role of histamine in the central nervous system, histamine functions as neurotransmitter. It is known that H₁ receptor is highly distributed in thalamus, hypothalamus, cerebellum and prosencephalon. These nerve cells regulate body temperature, ADH's secretion, blood pressure, drinking water, etc., all of which are mediated by H₁ and H₂ receptor.

Histamine which functions as shown above is released from mast cell by various kinds of drug as well as inflammation or allergic reaction. In therapeutic drugs, various kinds of alkaloid such as morphine, codeine, atropine, etc.; antibiotics; tubocurarine; succinylcholine; radiation contrast media; and plasma expander such as dextran, polyvinylpyrrolidone, etc. cause histamine release.

Histamine release can be inhibited by cAMP-increasing drug such as adrenergic agonist, various kinds of esterase-inhibiting substance, energy production enzyme-inhibiting substance (fluorine), chymotrypsin-inhibiting substance, etc. Cromolyn sodium stabilizes cell membrane of mast cell to inhibit release of histamine and leukotriene D₄ in bronchial mucosa, and so is used for the prevention of bronchial asthma attack.

Therefore, histamine is a primary mediator in allergic reaction, and functions solely or with other factors for asthma, rhinitis and skin disease such as urticaria and atopic dermatitis [Scannell R T et al., Mini Rev Med. Chem., 4(9) 923-33, 2004, Imaizumi A et al., J Dermatol Sci., 33(1) 23-9, 2003, Kapp A et al., J Drugs Dermatol., 3(6) 632-9, 2004]. Also, histamine affects cold, nausea and emesis, hyperacidity, gastroesophageal reflux disease, duodenal ulcer, inflammation, and hypothermia and hypotension related to anaphylaxis [Latsen J S., Pharmacotheraphy, 21: 28S-33S, 2001., Leurs R., Clin Exp Allergy 32(4) 489-98, 2002., Makabe-Kobayashi Y et al., J Allergy Clin Immunol., 110(2) 298-303, 2002.]. In order to prevent or treat these diseases, numerous drugs including diphenhydramine, tripelennamine, chlorpheniramine, meclizine, promethanzine, astemizole, etc. have been developed, and it was recently reported that these drugs are useful for nerve protection (dementia) and cognitive function increase [Bachurin S et al., Ann NY Acad. Sci., 939:424-35, 2001., Nakazato E. et al., Life Sci., 67(10) 1139-47, 2000].

The present inventors have continued to search natural products to find out substances having anti-histamine activity. As a result, they discovered that aloe, bamboo, rice plant, etc. have anti-histamine activity, and identified that the active ingredient isolated from the above natural substances is isoorientin, to complete the present invention.

### SUMMARY OF THE INVENTION

One object of the present invention is to provide a pharmaceutical composition for the prevention or treatment of diseases mediated by physiological change or functional disorder by excessive histamine comprising naturally-derived isoorientin.

Another object of the present invention is to provide a use of naturally-derived isoorientin for the manufacture of a medicament for the prevention or treatment of diseases mediated by physiological change or functional disorder by excessive histamine.

Another object of the present invention is to provide a method for preventing or treating diseases mediated by physiological change or functional disorder by excessive histamine in a subject, comprising administering a therapeutically effective amount of naturally-derived isoorientin to the subject.

### BRIEF DESCRIPTION OF THE DRAWINGS

FIG. 1 is ¹H-NMR spectrum of isoorientin.

FIG. 2 is ¹³C-NMR spectrum of isoorientin,

FIG. 3 is negative HPLC ESI-MS spectrum of isoorientin.

### DISCLOSURE OF THE INVENTION

To achieve the above objects, the present invention provides a pharmaceutical composition for the prevention or treatment of diseases mediated by physiological change or functional disorder by excessive histamine comprising naturally-derived isoorientin as an active ingredient.

The present invention also provides a use of naturally-derived isoorientin for the manufacture of a medicament for the prevention or treatment of diseases mediated by physiological change or functional disorder by excessive histamine.

The present invention also provides a method for preventing or treating diseases mediated by physiological change or functional disorder by excessive histamine in a subject, comprising administering a therapeutically effective amount of naturally-derived isoorientin to the subject.

In the present invention, “diseases mediated by physiological change or functional disorder by excessive histamine” refer to allergic disease, asthma, rhinitis, atopic disease, skin disease, cold, hyperacidity, gastroesophageal reflux disease, duodenal ulcer, inflammation, and nervous system disorder, including atopic dermatitis, urticaria, asthma, dementia, etc.

In the present invention, “allergic disease” refers to urticaria, nausea, emesis, atopic dermatitis, anaphylaxis, asthma, rhinitis, etc., and “nervous system disorder” refers to dementia, cognitive function decrease, etc.

In the present invention, it is preferable that the composition comprising isoorientin is particularly aloe, bamboo or rice plant extract.

Preferably, the aloe, bamboo or rice plant extract comprising isoorientin is, but not limited to, extract of water, or C₁₋₄ alcohol such as methanol, ethanol, propanol, butanol, etc., or mixed solvent thereof. In particular, the aloe extract comprising isoorientin is preferably obtained by extracting aloe with 30-80% methanol or ethanol. The bamboo extract comprising isoorientin is preferably obtained by extracting bamboo with water to obtain dehydrated extract, and re-extracting said dehydrated extract with methanol or ethanol. The extract includes a whole extract and its fraction. In addition, the aloe extract comprising isoorientin is preferably obtained from, but not limited to, rind of aloe.

The composition of the present invention can be prepared into conventional pharmaceutical preparations according to conventional methods in the pharmaceutical field, for example, solution such as drinks, syrup, capsule, granule, tablet, powder, pill, ointment, emulsion, gel, skin external preparation such as cream, etc., by optionally mixing it with pharmaceutically acceptable carrier, excipient, etc.; and can be administered orally or parenterally. Preferably, the composition of the present invention may be orally administered in capsule, tablet, and drink, before or after a meal for quick effect.

Capsule, tablet, powder, granule, solution, pill, gel, etc. comprising the composition of the present invention are preferably used as medicine or health care products. In the present invention, “health care products” mean food products prepared and processed in the form of tablet, capsule, powder, granule, solution, pill, gel, etc., by using material or ingredients having useful function to the human body.

The composition of the present invention is appropriately administered depending on the extent of absorption of active ingredients into the body; excretion rate; age, weight, sex, and condition of patient; severity of treated disease; etc. However, generally, it is preferable to administer the present composition to adult by 0.01-500 mg/kg, preferably 0.1-200 mg/kg, per day, 1˜3 times a day.

Hereinafter, the present invention will be described in more detail with reference to the following Examples and Experimental Examples, but the scope of the present invention should not be construed to be limited thereby in any manner.

### EXAMPLE 1

### Extraction and Identification of Isoorientin

1. Isolation of Anti-Histamine Active Ingredient

The present inventors tried to select a fraction with best yield and activity among extracts of natural products, and to isolate anti-histamine active ingredient from the fraction. The extracts of natural products were evaporated under reduced pressure, and well dissolved in a small quantity of water. Then, the extracts were fractionated with an equivalent amount of CH₂Cl₂ to remove non-polar materials, and fractionated with an equivalent amount of BuOH. Since desired isoorientin is present in BuOH layer, the BuOH layer was evaporated under reduced pressure, and then Silica column was carried out thereto. A mixed solvent of CHCl₃, MeOH and water was used in the initiation ratio of C:M:W=7:3:1 up to 6:4:1.

Among Silica column fractions, the fraction containing isoorientin was evaporated under reduced pressure, and Sephadex LH20 column was carried out thereto. 100% MeOH was used as elution solvent. Among Sephadex LH20 column fractions, isoorientin was obtained by TLC and HPLC analysis.

2. Identification of Active Ingredient

Yellow powder C₂₁H₂₂O₁₁; ¹H-NMR (300 MHz, d6-DMSO) and ¹³C-NMR (75 MHz, d6-DMSO) data was compared with Reference. Refer to Biosci. Biotechnol. Biochem., 67(2), 410-414, 2003.

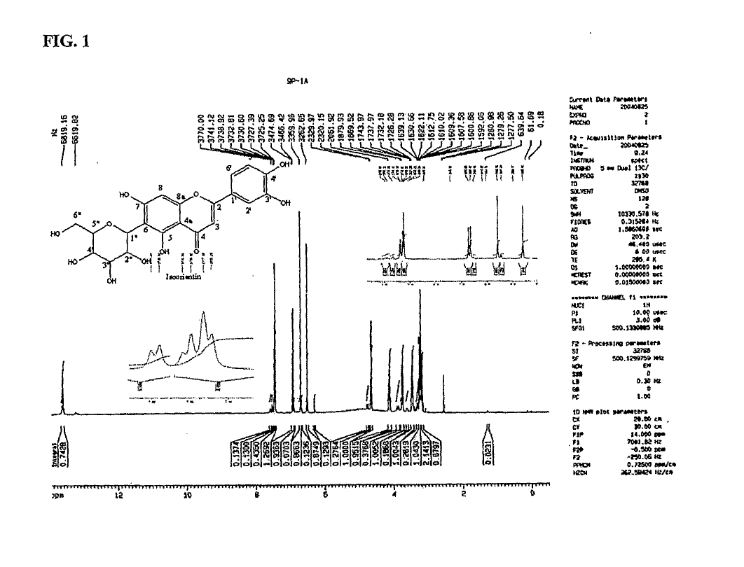

Through in vitro analysis, a compound having anti-histamine activity was isolated from aloe, rice plant and bamboo fractions by pursuing activity. NMR spectroscope was used to identify the structure of the isolated compound. In ¹H-NMR spectrum (FIG. 1 and Table 1), a single peak for one proton was observed at δ 13.64 ppm, and most peaks observed in this region are resulted from shifting to low magnetic field by hydrogen bond. At δ 7.47 ppm, doublet (J=8.3 Hz) by ortho-coupling with adjacent δ 6.97 ppm, and doublet (J=2.2 Hz) by meta-coupling with δ 7.45 ppm were observed. In addition, single peaks for one proton each were observed at δ 6.72 ppm and δ 6.53 ppm. At δ 4.65 ppm, a typical anomeric proton has the coupling constant value of J=9.8 Hz as doublet, and so it was observed that glucose is bound to.

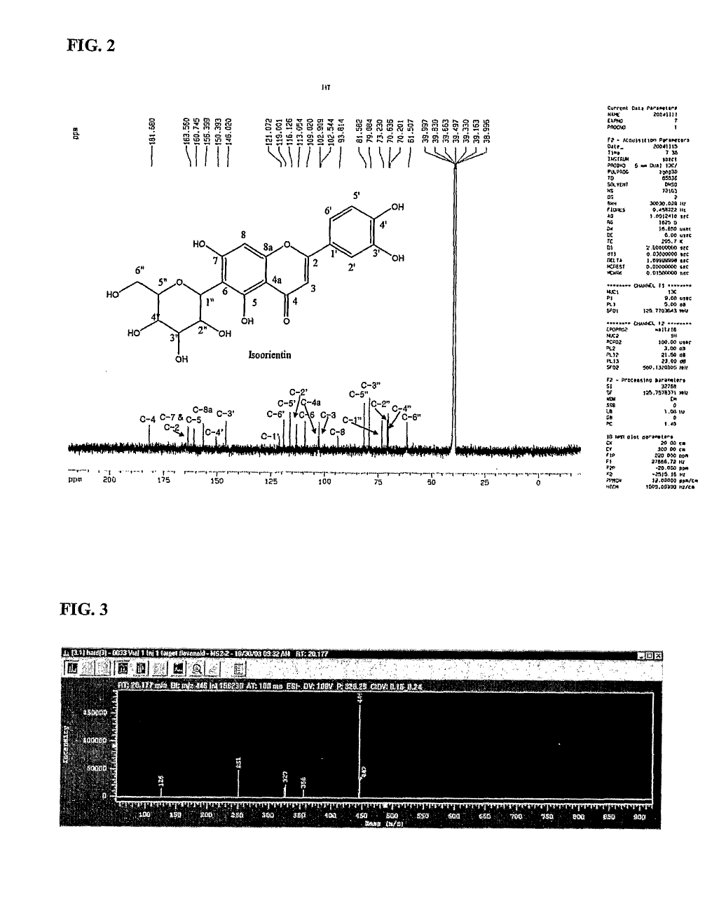

In ¹³C-NMR spectrum (FIG. 2), total 21 carbons were observed, in particular, carbonyl carbon at δ 182.1 ppm, and anomeric carbon at δ 73.5 ppm. In negative HPLC ESI-MS spectrum (FIG. 3), parent ion peak was observed at m/z 487, and so the molecular weight was anticipated as m/z 488.

TABLE 1
¹H and ¹³C NMR chemical movement against isoorientin

| | HT ppm)^{†} | | Reference ppm)^{a} | |
|---|---|---|---|---|
| Position | ¹H ppm) | ¹³C ppm) | ¹H ppm) | ¹³C ppm) |
| 4 | | 182.0 | | 182.3 |
| 2 | | 163.9 | | 164.2 |
| 7 | | 163.9 | | 164.1 |
| 5 | 13.64 (1H s 5-OH | 161.1 | 13.53 (1H s 5-OH | 161 |
| 8a | | 156.7 | | 156.8 |
| 4′ | | 150.7 | | 150.3 |
| 3′ | | 146.4 | | 146.2 |
| 1′ | | 121.4 | | 121.8 |
| 6′ | 7.47 (1H dd, J 8 3 2 2 Hz | 119.3 | 7.42 (1H d J 7 9 Hz | 119.5 |
| 5′ | 6.97 (1H d J 8 3 Hz | 116.5 | 6.93 (1H d J 7 9 Hz | 116.5 |
| 2′ | 7.45 (1H d J 2 2 Hz | 113.4 | 7.41 (1H s | 113.5 |
| 6 | | 109.4 | | 109.2 |
| 4a | | 103.2 | | 103.7 |
| 3 | 6.72 (1H s | 102.9 | 6.57 (1H s | 103.2 |
| 8 | 6.53 (1H s | 94.1 | 6.53 (1H s | 94.2 |
| 5″ | | 81.9 | | 81.8 |
| 3″ | | 79.4 | | 79.3 |
| 1″ | 4.65 (1H d J 9 8 Hz | 73.6 | 4.62 (1H d J 9 8 Hz | 73.5 |
| 2″ | | 71.0 | | 70.9 |
| 4″ | | 70.5 | | 70.7 |
| 6″ | | 61.8 | | 61.9 |

Based on the above instrumental analysis results and a relevant Reference [Abdul Mun'IM, Osamu Negishi, and Testuo Ozawa.(2003), Antioxidative Compounds from Crotalaria sessiliflora., Biosci. Biotechnol. Biochem., 67(2), 410-414], the compound having anti-histamine activity isolated from the extracts was identified as isoorientin.

### EXAMPLE 2

### Search for Plants Which Contain Isoorientin, and Content Analysis

In the Example 1 above, it was confirmed that isoorientin has anti-histamine activity. Thus, in this Example, analysis was carried out for some plant extracts which Applicant owns. The following Table 2 shows the plant extracts and their contents of isoorientin.

To analyze the extracts, HITACHI system (pump: L-7100, detector: L-7455, interface: D-7000, column oven: L-7300, automatic sampler: L-7200) was used as HPLC under the analysis conditions that the stationary phase is Phenomenex C18 4.6×250 mm; the mobile phase is gradient condition (solvent A: acetonitrile, and solvent B: 0.1% H₃PO₄ in water); the flow rate is 1.5 mL/min; the total analysis time is 85 min; the temperature of column oven is 35° C.; the concentration of sample is 50,000 ppm; the input amount is 10 μl; and UV detector at 330 nm is used.

TABLE 2
Isoorientin content analyzed from the
plants (ingredient content %/yield %)

| | Isoorientin |
|---|---|
| Name of the plants | content %/yield % |
| Phyllostachys nigra var. henonis | 0.31/9.9 |
| Phyllostachys pubscense | 0.25/10.97 |
| Phyllostachys bambusoides | 0.4/11.69 |
| Sasa coreana | 0.08/8.82 |
| Sasa borealis | 0.52/14.2 |
| Oryza alta | 0.49/13.8 |
| Phyllostachys heterocycla var. pubescens | 0.8/11.3 |
| Phyllostachy nigra | 0.46/10.9 |
| Phyllostachys nigra var. henonis Stapf | 0.62/10.4 |
| Phyllostachys bambusoides var. | 0.39/10.7 |
| castillonis-inversa Houzeau de Lehaie | |
| Arundinaria graminea Makino | 1.05/11.9 |
| Phyllostachys aurea Carriere ex A. Riviere | 0.26/10.3 |
| et C. Riviera | |
| Phyllostachys bambusoides var. tanakae Makino | 0.18/8.7 |
| Pseudosasa japonica | 0.19/7.38 |
| Sasa borealis var. gracilis | 0.23/6.36 |
| Lophatherum gracile | 0.24/10 |
| aloe vera | 0.18/25 |

### EXAMPLE 3

### Preparation of Fraction with High Isoorientin Content from Aloe

Aloe vera rind of 1 kg was extracted with 15 L of 95%, 80%, 70%, 60%, 50%, 40% or 30% ethanol, and evaporated under reduced pressure to give hydrated extract. Isoorientin content of the obtained extract was analyzed by HPLC in the same manner of the Example 2 above. As a result, it was shown that the isoorientin content was highest in 50% ethanol extract.

TABLE 3
Content and yield of isoorientin depending on ethanol
content of extract solvent of aloe by parts

| Part | Extract Solvent | Isoorientin content %/yield % |
|---|---|---|
| Rind | 95% ethanol | 0.15/2.9 |
| | 80% ethanol | 0.38/3.4 |
| | 70% ethanol | 0.53/4.1 |
| | 60% ethanol | 0.82/4.8 |
| | 50% ethanol | 1.2/5.4 |
| | 40% ethanol | 0.73/6.4 |
| | 30% ethanol | 0.27/7.8 |
| Gel | 95% ethanol | 0.03/9.9 |
| | 80% ethanol | 0.08/10.97 |
| | 70% ethanol | 0.13/11.69 |
| | 60% ethanol | 0.15/15.2 |
| | 50% ethanol | 0.05/36.7 |
| | 40% ethanol | 0.01/41.4 |
| | 30% ethanol | 0.01/62.3 |

### EXAMPLE 4

### Preparation of Fraction with High Isoorientin Content from Bamboo

Bamboo leaves of 10 kg were extracted with 150 L of water at 80° C. for 8 hours, and evaporated under reduced pressure to give 680 g of extract. 500 g of the hydrated extract was extracted with 4 L of ethanol at 70° C. for 2 hours, cooled to room temperature, and filtered. The filterate was evaporated under reduced pressure to give 127 g of concentrated extract. 100 g of the concentrated extract was added with 800 ml of water, extracted at 80° C. for 2 hours, and filtered. The filterate was lyophilized to give 61 g of hydrated extract.

HPLC analysis according to the analysis method of the Example 2 indicated that the isoorientin content was 3.2%.

### EXPERIMENTAL EXAMPLE 1

### Measurement of Histamine Release Inhibition Activity of Isoorientin

1. Purification of Guinea Pig Lung Mast Cell

Lung tissues (3 g/1 guinea pig) were isolated from 8 guinea pigs (female, 200 g), fat tissue, bronchus and blood were removed therefrom, and treated with enzyme (5 mg/ml collagenase, 1.8 unit/27 ul elastase) three times by using Tyrode buffer (TGCM buffer) containing Ca²⁺, Mg²⁺ and 0.1% gelatin for 15, 15 and 25 minutes. In each enzyme treatment, the lung tissues were filtered with nylon mesh and metal mesh (100 μm), and centrifuged (called as monodispersed mast cells). Pellets were suspended in 16 ml of buffer (TG buffer) containing Ca²⁺, Mg²⁺-free, and 0.1% gelatin, loaded to rough Percoll (1.041 mg/ml density), and centrifuged at 1,400 rpm for 25 minutes to give pellets. The cells were suspended again in 8 ml of TG buffer, loaded to discontinuous Percoll (1.06-1.10 mg/ml density), and centrifuged again at 1400 rpm for 25 minutes to afford various kinds of cell layers. Among them, mast cells were mainly present in 3^{rd} or 4^{th} layer, and so cells obtained from these layers were washed with TGCM buffer twice. Total cells and mast cells were dyed with trypan blue and alcian blue, respectively, and cell numbers were measured by microscope to determine the purity of mast cells, whereby the purity was confirmed as about 80˜90%.

2. Assay of Histamine Released from Mast Cell Activated with Antigen/Antibody Reaction

Mast cells (4×10⁵ cells) were treated with guinea pig IgG1 antibody (anti-OVA 1 ml/10⁶ cells), reacted at 37° C. for 45 minutes, and then washed with TGCM buffer to remove anti-OVA antibody which was not bound to mast cells membrane. The cells were suspended in 1 ml of TGCM buffer, and treated with drug (testing substance) at each concentration for 5 minutes. The suspension was sensitized with 1.0 μg/ml OVA (ovalbumin), reacted for 10 minutes, cooled at ice, and centrifuged. After the centrifuge, histamine in supernatant was measured.

The amount of histamine released in each sample was measured by using the automated continuous-flow extraction and fluorometric analyzer (Astoria analyzer series 300, Astoria-pacific international, Oragon, USA) which is modified from method (1) of Siraganian. 1N-hydrochloric acid, 0.73M phosphoric acid, 5N sodium hydroxide, 1N sodium hydroxide, saline diluent and sampler wash, and o-phthalaldehyde solution were prepared, a tube connected to the analyzer was connected, and histamine stock solution was diluted to 20 ng, long, 5 ng, 3 ng and 1 ng to obtain a standard curve of concentration-dependent result. Each sample was diluted with 2% perchloric acid to measure the amount of histamine. The amount of histamine contained in each sample was calculated as percentage against the amount of histamine contained in total cells used, as follows.

$${}^{*} Amount of histamine = \frac{histamine release amounts in sample - spontaneous release}{Total histamine release amounts - spontaneous release} \times 100$$

The above measurement results were shown in the following Table 4. Reviewing anti-histamine activity of isoorientin isolated from the natural products, it was confirmed that isoorientin inhibited histamine release in mast cells in a concentration-dependent way, and the IC₅₀ value was 30 μg.

TABLE 4
Effect of isoorientin on the histamine release from passively
sensitized (anti-OVA antibody) lung mast cells activated by
1.0 μg/4 × 10⁵ cells^{a}

| Name of the samples | Histamine (%) |
|---|---|
| OVA | 32.5 ± 0.86 |
| Isoorientin (1.25 μg) | 27.9 ± 2.62 (14)** |
| Isoorientin (2.5 μg) | 25.6 ± 1.49 (21.3)** |
| Isoorientin (5 μg) | 21.9 ± 2.01 (32.6)* |
| Isoorientin (10 μg) | 17.2 ± 1.07 (47.2)** |
| 95% ethanol extract of rind in Example 3 (50 μg) | 24.7 ± 1.75 (24.0)** |
| 70% ethanol extract of rind in Example 3 (50 μg) | 15.2 ± 0.93 (53.2)*** |
| 50% ethanol extract of rind in Example 3 (50 μg) | 5.2 ± 0.43 (84.0)** |
| 30% ethanol extract of rind in Example 3 (50 μg) | 20.4 ± 1.75 (37.2)** |
| Bamboo extract in Example 4 (50 μg) | 19.2 ± 0.82 (40.9)** |
| Bamboo extract in Example 4 (100 μg) | 3.2 ± 0.52 (90.15)** |

^{a}Guinea pig mast cells were isolated, and purified by enzyme digestion, and rough and discontinuous percoll density gradient method. Mast cells (4 × 10⁵ cells) were passively sensitized by anti-OVA antibody, and challenged by 1.0 μg/ml OVA. Isoorientin was added 5 min before antigen challenge. Histamine in supernatant was determined by fluorometric analyzer.
^{b}The amount of histamine released was expressed as the percentage of the total histamine content. Parenthesis was expressed as a decreasing percentage evoked by UG4-92 pretreatment.
*p < 0.05;
**p < 0.01;
***p < 0.001 compared with OVA alone.
( ): inhibition %

Formulation Example 1: Preparation of Solution

| Isoorientin | 1 | g |
|---|---|---|
| Sugar | 10 | g |
| Isomerized sugar | 10 | g |
| Smell of lemon | proper quantity | |
| Total amount after adding purified water | 100 | ml |

The above-mentioned ingredients were mixed according to conventional preparation method for solution, and sterilized to give a solution.

Formulation Example 2: Preparation of Capsule

| | |
|---|---|
| Isoorientin | 500 mg |
| Lactose | 50 mg |
| Starch | 50 mg |
| Talc | 2 mg |
| Magnesium Stearate | proper quantity |

The above-mentioned ingredients were mixed, and filled in a gelatin capsule according to conventional preparation method for capsule to give a capsule.

### INDUSTRIAL APPLICABILITY

The composition comprising isoorientin, use of isoorientin and prevention or treatment method using isoorientin according to the present invention show excellent histamine suppression effects, and so can be used for the prevention or treatment of various kinds of allergic disease, atopic disease, skin disease, cold, hyperacidity and nervous system disorder.

What is claimed is:

1. A pharmaceutical composition for the prevention or treatment of diseases mediated by physiological change or functional disorder by excessive histamine comprising naturally-derived isoorientin as an active ingredient.

2. The composition of claim 1, wherein the diseases mediated by physiological change or functional disorder by excessive histamine are allergic disease, atopic disease, skin disease, cold, hyperacidity or nervous system disorder.

3. The composition of claim 1, wherein the composition comprising naturally-derived isoorientin is aloe, bamboo or rice plant extract.

4. The composition of claim 3, wherein the aloe extract comprising isoorientin is obtained by extracting aloe with 30-80% methanol or ethanol.

5. The composition of claim 3, wherein the bamboo extract comprising isoorientin is obtained by extracting bamboo with water to obtain dehydrated extract, and re-extracting said dehydrated extract with methanol or ethanol.

6. The composition of claim 3 or 4, wherein the aloe extract comprising isoorientin is obtained from rind of aloe.

7. A use of naturally-derived isoorientin for the manufacture of a medicament for the prevention or treatment of diseases mediated by physiological change or functional disorder by excessive histamine.

8. The use of claim 7, wherein the diseases mediated by physiological change or functional disorder by excessive histamine are allergic disease, atopic disease, skin disease, cold, hyperacidity or nervous system disorder.

9. The use of claim 7, wherein the naturally-derived isoorientin is aloe, bamboo or rice plant extract.

10. The use of claim 9, wherein the aloe extract comprising isoorientin is obtained by extracting aloe with 30-80% methanol or ethanol.

11. The use of claim 9, wherein the bamboo extract comprising isoorientin is obtained by extracting bamboo with water to obtain dehydrated extract, and re-extracting said dehydrated extract with methanol or ethanol.

12. The use of claim 9 or 10, wherein the aloe extract comprising isoorientin is obtained from rind of aloe.

13. A method for preventing or treating diseases mediated by physiological change or functional disorder by excessive histamine in a subject, comprising administering a therapeutically effective amount of naturally-derived isoorientin to the subject.

14. The method of claim 13, wherein the diseases mediated by physiological change or functional disorder by excessive histamine are allergic disease, atopic disease, skin disease, cold, hyperacidity, or nervous system disorder.

15. The method of claim 13, wherein the naturally-derived isoorientin is aloe, bamboo or rice plant extract.

16. The method of claim 15, wherein the aloe extract comprising isoorientin is obtained by extracting aloe with 30-80% methanol or ethanol.

17. The method of claim 15, wherein the bamboo extract comprising isoorientin is obtained by extracting bamboo with water to obtain dehydrated extract, and re-extracting said dehydrated extract with methanol or ethanol.

18. The method of claim 15 or 16, the aloe extract comprising isoorientin is obtained from rind of aloe.
